(19) United States
(12) Patent Application Publication
(10) Pub. No.: US 2005/0186155 A1
(43) Pub. Date: Aug. 25, 2005

(54) Particles that include a solid coating and a liquid core, preparations comprising these particles and processes for preparing these particles

(75) Inventors: Thomas Raschke, Pinneberg (DE); Frank Hetzel, Welle (DE); Volker Kallmayer, Hamburg (DE); Stefan Wiesner, Hamburg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/992,622

(22) Filed: Nov. 18, 2004

(30) Foreign Application Priority Data
Nov. 18, 2003 (DE) 103 53 780.5

(51) Int. Cl. A61K 7/42; A61K 7/06; A61K 9/16

(52) U.S. Cl. 424/59; 424/490; 264/4

(57) ABSTRACT

The present invention is a particle, comprising a solid coating primarily comprising at least one wax; and a liquid core primarily comprising at least one polyol, wherein the water content of the core is at most 50% by weight, based on the total weight of the core. The present invention also includes cosmetic or dermatological preparations comprising a plurality of the particles of the invention and processes for preparing the particles of the invention.

### FIELD OF THE INVENTION

The present invention relates to a novel administration form for moisture-sensitive or sparingly water-soluble active ingredients, i.e. those which are soluble in polyols but not in water, to a process for their preparation, and to cosmetic and/or dermatological preparations, in particular cosmetic and/or dermatological preparations, which comprise these.

### BACKGROUND OF THE INVENTION

When moisture-sensitive active ingredients are used, there is the fundamental and obvious problem of incorporating such active ingredients into aqueous preparations. In the case of polar, but water-insoluble active ingredients with a high crystallization tendency, there is the additional problem, at least for topical application, of supplying the active ingredient on the skin in a bioavailable form. An active ingredient which is ready on the skin in crystallized form in an aqueous preparation will generally not be bioavailable and not penetrate into the skin.

Much has been proposed to solve this problem. Although a large number of solutions are not aimed at topical application, they are conceivable for such an application. However, in practice it is evident that topical application places different requirements on such solutions than a parenteral or oral application. By way of example, it may be mentioned that in the case of topical application, the active ingredient in question has to overcome the skin barrier.

The specification EP 779071 discloses W/O/W emulsions in which a water-sensitive topical active ingredient is present in one of the water phases which has a water activity of at most 0.85. However, nothing is disclosed about systems which are at least partly solid.

The specification EP 755674 discloses topical preparations comprising ascorbic acid and polyol, the amount of which is such that the water activity is at most 0.85, and an additional oily or polymeric structuring agent is present. However, nothing is disclosed about systems which are at least partly solid.

Both of the specifications cited start from polyol-rich, but essentially aqueous phases, and not from pure polyol phases. For the purposes of this application, a pure polyol phase is understood as meaning a phase for whose preparation no water has been used, but instead polyols in their standard commercial forms. This permits, in particular, residual water contents of up to 15 per cent by mass.

The publication Adv. Drug Deliv. Rev. 54, Suppl 1 (2002), pages S131 to S155 discloses so-called solid lipid nanoparticles (SLN) and nanostructured lipid carriers (NLC). These are essentially wax particles into which fat-soluble active ingredients can be incorporated in solid form. By contrast, nothing is disclosed about active ingredients dissolved in a hydrophilic (liquid) phase which are incorporated in some way into the particles.

The publication Adv. Drug Deliv. Rev. 47 (2001), S165-196 likewise discloses SLN systems. This describes the diverse ways in which suspensions of solid lipid particles differ from the corresponding emulsions of liquid lipid particles, i.e. droplets. Thus, by virtue of correspondingly developed solid lipid phases it is possible, for example, to improve the release kinetics and the stability of oxidation-sensitive active ingredients in a decisive manner.

EP 1151741 discloses microcapsules with an aqueous core and polymeric and/or wax-like coating for formulating water-soluble active ingredients. In contrast to this, the present invention describes microcapsules with essentially nonaqueous cores whose main constituent is polyols. By virtue of this, it is possible, in contrast to the processes of the prior art, to also formulate water-sensitive and even water-insoluble active ingredients.

### SUMMARY OF THE INVENTION

Starting from this, the object of the present invention was to find particles containing moisture-sensitive active ingredients which permit good stabilization of the active ingredients coupled with particularly good topical active ingredient release. It has not proven foreseeable for the person skilled in the art that particles comprising a solid coating and one or more liquid cores, where the coating consists primarily of wax and the core or cores consist primarily of at least one polyol, and the water content of the core or cores is at most 50% by weight, based on the total composition of the core or cores, overcome the shortcomings of the prior art. Such particles permit the preparation of water-rich preparations with moisture-sensitive active ingredients and thus a particularly pleasant feel on the skin in the case of the application of topical preparations prepared therewith. In addition, the particles or the cores within the particles do not coalesce as is often observed with the droplets of triple emulsions. The active ingredients present in the particles can be incorporated into water-rich formulations in dissolved or stabilized form.

It has also been found that it is preferred if the average diameter of the particles is 1 to 50 μm, particularly preferably 10 to 30 μm. These particularly small particles have a larger wax-water interface, which accelerates the diffusion of water into the core and thus negates the advantages of the particles according to the invention. By contrast, larger particles impair the feel on the skin upon application.

It is preferred if the polyol is chosen from the group of unbranched polyhydroxyalkanes, where glycerol, butylene glycol, dipropylene glycol and pentanediol are particularly preferred. These two polyols are still polar enough not to be miscible with the wax. They have the added benefit of skin moisturization and are very well tolerated by the skin.

It is also preferred if the wax has a melting point of from 35 to 80° C., particularly preferably from 40 to 60° C. This has the advantage that the wax, following application, can be dissolved in the skin lipids or be distributed by shearing and thus release the active ingredient. Waxes with a higher melting point will do this too slowly and are, moreover, problematical in the production of the wax particles. Waxes with a low melting point are in some cases even partially liquid during storage, resulting in poorer shielding against the diffusion of water.

It is further preferred if the wax is hydrophobic wax, particularly preferably a paraffinic wax. Due to the poor solubility of water in the wax, hydrophobic waxes slow the diffusion of water into the core particularly well.

It is particularly preferred if TiO₂ is additionally present at the interface between coating and core. In the preparation process, TiO₂ stabilizes the emulsion, in the product it additionally shields the core so to speak by immuring against penetrating water and UV radiation.

It is further particularly preferred if an emulsifier is present, either instead of or in combination with TiO₂. Emulsifiers reduce the interfacial energy and thus considerably facilitate the preparation of the polyol-in-wax emulsion.

It is likewise preferred if at least one moisture-sensitive cosmetic and/or dermatological active ingredient is additionally present in the core, in particular a polar, but water-insoluble active ingredient with a high crystallization tendency or an active ingredient which decomposes under the influence of moisture. Particles according to the invention are particularly preferably suitable for stabilizing ingredients of liquorice, such as, for example, glycirrhizinic acid, isoflavonoids, such as, for example, genistein or daidzein, creatine, carnosine, pentacyclic triterpenes, such as, for example, sericosides or ursolic acid, flavonoids, such as, for example, rutin or quercetin, green tea active ingredients, such as, for example, epigallocatechin gallate, phytosterols, such as, for example, β-sitosterol or campesterol, octadecenedioic acid, steroids, such as, for example, cholesterol or dehydroepiandrosterone, and dihydroxyacetone. It is likewise preferred if an active ingredient which decomposes under the influence of moisture chosen from the group consisting of acetylcarnitine, acetylsalicylic acid, peptides consisting of 3-8 amino acids, enzymes, such as, for example, serine proteases, lipases, telomerases, DNA repair enzymes and ascorbic acid or derivatives thereof is additionally present.

It is particularly preferred if at least one antioxidant, particularly preferably ubiquinone, ubiquinol, vitamin E, EDTA, butylhydroxytoluene, α-tocopherol, ascorbyl palmitate, is additionally present. Inter alia, these complex metal ions which catalyze decomposition of ingredients.

It is further preferred if at least one photoprotective filter is additionally present in the coating, particularly preferably butylmethoxydibenzoylmethane or a filter from the group of anisotriazines. UV-sensitive active ingredients can thus be stabilized since the filter is present in concentrated form at the correct place and not, as is usual, within the entire resulting preparation. Thus, virtually UV-filter-free preparations are obtainable in which the active ingredients are protected against UV light, although the preparation does not have to have a noteworthy photoprotective effect (SPF=0-3).

The invention also covers cosmetic and/or dermatological preparations comprising the described particles and two alternative processes for the preparation of these particles:

a first process for the preparation of the particles according to the invention, where
(a) the wax is firstly dissolved in a volatile organic solvent,
(b) a polyol-in-solvent emulsion is prepared from this organic phase and an active ingredient-containing polyol phase,
(c) a polyol-in-solvent-in-water emulsion is prepared from this preemulsion and a water phase,
(d) the solvent is removed,
(e) the particles are separated off from the resulting polyol-in-wax-in-water suspension,
   and a second process for the preparation of particles according to the invention, where
(a) a polyol-in-wax preemulsion is prepared above the melting temperature of the wax from an active ingredient-containing polyol phase and a wax phase,
(b) a polyol-in-wax-in-water emulsion is prepared above the melting temperature of the wax phase from this preemulsion and a water phase,
(c) a polyol-in-wax-in-water suspension is prepared by cooling below the melting temperature of the wax phase from the polyol-in-wax-in-water emulsion, and
(d) the particles are optionally separated off from the polyol-in-wax-in-water suspension.

The omission of an individual constituent can adversely affect the unique properties of the overall composition. Therefore, all of the constituents of the preparations according to the invention stated are preferably included to carry out the invention.

### DETAILED DESCRIPTION OF THE INVENTION

For the purposes of the present specification, particles are any shaped particles which are solid due to their coating. They can have various shapes, for example that of a sphere, an ellipsoid, a torus or ring, a cube of greater or lesser deformation, cylinder or tetrahedron, or else be irregularly shaped, for example in the form of a sphere cluster or provided in some other way with an irregular surface.

The average diameter for the purposes of the present specification is the number-average—provided a distribution of the diameters arises—of the individual diameters measured. The particle sizes can be measured using scatter methods (photon correlation spectrometry) or light or electron microscopic methods (optical reflected-light or transmitted-light microscopy, transmission electron microscopy).

For the purposes of the present specification, “solid coatings” completely surround a core and consist of a material which is solid at customary room or storage temperatures. It may be crystalline or glass-like. They have a wall thickness which—despite the difficulties in stating a wall thickness which are known and can be attributed to the possible presence of two or more polyol drops in one capsule—is greater than 500 nm. Part of the coating can also be solid substances. The coatings determine the shape of the particles.

For the purposes of the present specification, a solid substance is understood as meaning a substance which is in a form below its melting point. Objects manufactured from such solid substances may also be solid. Substances or objects in a form below their melting point which have a certain deformability, such as elastic bodies (e.g. rubber spheres) or very slow-flowing amorphous substances, such as waxes or glasses, are also solid. Liquid-crystalline objects, by contrast, are not solid for the purposes of the present specification.

Here, wall thickness is the shortest distance between the phase boundary between coating and surrounding medium or vacuum on the one hand and the phase boundary between coating and core on the other hand.

Liquid cores for the purposes of the present specification are found within the solid coating. They are liquid at customary room or storage temperatures, but primarily at the application and body temperature.

Waxes for the purposes of the present specification are water-insoluble plastic substances solid at room temperature which are reversibly liquid above a substance-specific temperature. From a chemical point of view, they can, for example, consist of
fatty alcohols
fatty acids
(ester waxes) esters of fatty acids and fatty alcohols, where the fatty acids or fatty alcohols may, independently of one another, be saturated or unsaturated, branched or unbranched,
(glyceride waxes) esters of saturated or unsaturated, branched or unbranched fatty acids with polyols, where, in a molecule, various acid residues may be present and not all of the hydroxy groups of the polyol have to be esterified, or
(microcrystalline waxes, ceresines, ozokerites) from branched, linear or cyclic hydrocarbon compounds.

Waxes can also consist of a mixture of any number of different compounds from one or more of the above-described groups. Examples of customary waxes are.

Polyols for the purposes of the present specification are molecules which contain two or more hydroxy groups, for example glycerol (propane-1,2,3-triol), propylene glycols (propane-1,2-diol, propane-1,3-diol), butylene glycols (butane-1,2-diol, butane-1,3-diol, butane-1,4-diol, butane-2,3-diol), ethylene glycol (ethane-1,2-diol), dipropylene glycol, pentane-1,2-diol and also ethers thereof (polyglycerols, polyethylene glycols, polypropylene glycols, polyethylene-polypropylene copolymers).

A hydrophobic wax for the purposes of the present specification is a wax in which an especially small amount of water can dissolve and which does not swell in aqueous media. These are, in particular, triglyceride waxes, ester waxes and hydrocarbon waxes, such as ceresine, ozokerite and microcrystalline wax.

A paraffinic wax for the purposes of the present specification consists of more than 95% by weight of saturated hydrocarbons.

TiO₂ for the purposes of the present specification are particles which consist essentially of titanium dioxide and have a size of between 10 nm and 50 μm, preferably between 20 nm and 1 μm, particularly preferably between 50 nm and 500 nm. The titanium dioxide may be present in any modification (rutile, anatase) and be doped with cations other than titanium. The particles can, moreover, be coated.

Emulsifiers for the purposes of the present specification are compounds which consist of a lipophilic and a hydrophilic moiety and are suitable for reducing the energy of an interface between two immiscible phases.

A cosmetic and/or dermatological active ingredient has an effect of a cosmetic or dermatological nature, such as wrinkle smoothing, cell protection, reduction in skin irritations, skin moisturization, improvement of the skin barrier, reduction in sebum production, increase in barrier lipid production, change in skin colour. Examples of such active ingredients are ascorbic acid, ascorbyl phosphate and other vitamin C derivatives, flavonoids, such as rutin or quercetin, and isoflavonoids, such as genistein or daidzein, dihydroxyacetone, acetylcarnitine, acetylsalicylic acid, peptides consisting of 3-8 amino acids, enzymes, such as, for example, serine proteases, lipases, telomerases and DNA repair enzymes, glycyrrhizinic acid, creatine, carnosine, pentacyclic triterpenes, such as, for example, sericosides or ursolic acid, green tea active ingredients, such as, for example epigallocatechine gallate, phytosterols, such as, for example, β-sitosterol or campesterol, octadecenedioic acid, steroids, such as, for example, cholesterol or dehydroepiandrosterone.

A moisture-sensitive active ingredient, in particular a cosmetic and/or dermatological active ingredient for the purposes of the present specification is either degraded in aqueous solution considerably more rapidly than in the undissolved state, for example by hydrolysis or oxidation (an active ingredient which decomposes under the influence of moisture), or crystallizes upon contact with water from a solution (polar, but water-insoluble active ingredient with high crystallization tendency). It can thus not be used directly in cosmetic and/or dermatological preparations if these are aqueous, which is the case for most cosmetic and/or dermatological preparations, since these are mostly W/O or O/W emulsions or even lipid-free or low-lipid aqueous preparations.

A polar but water-insoluble active ingredient with high crystallization tendency for the purposes of the present specification may be dissolved in polar, water-miscible liquids with the exception of water itself. However, if the polar liquid is diluted with water, then the mixing liquid is not a good solvent for the active ingredient and it crystallizes out. It generally has a high melting point. Due to its high melting point, it does not result in the formation of a liquid or liquid-crystalline phase, but in the precipitation of crystals. Such active ingredients may be flavonoids, such as rutin or quercetin, or isoflavonoids, such as genistein or genistein, liquorice ingredients, such as, for example, glycyrrhizinic acid, creatine, carnosine, pentacyclic triterpenes, such as, for example, sericosides or ursolic acid, green tea active ingredients, such as, for example, epigallocatechin gallate, phytosterols, such as, for example, β-sitosterol or campesterol, octadecenedioic acid, steroids, such as, for example, cholesterol or dehydroepiandrosterone.

For the purposes of the present specification, an active ingredient which decomposes under the influence of moisture undergoes chemical degradation or rearrangement as a result of the influence of moisture. The substance may undergo hydrolysis, polymerization, cyclization, rearrangement, or undergo oxidation or other destructive processes. Such active ingredients may be ascorbic acid and its derivatives or guanidine derivatives, such as creatine or carnitine esters, such as, for example, acetylcarnitine, acetylsalicylic acid, dihydroxyacetone, peptides consisting of 3-8 amino acids, enzymes, such as, for example, serine proteases, lipases, telomerases and DNA repair enzymes.

For the purposes of the present specification, an antioxidant prevents oxidation reactions, as can be caused by light or the ingress of oxygen. Such antioxidants may be ascorbic acid and its derivatives, tocopherol and other compounds of the vitamin E group, and their esters, toluene derivatives, such as di-tert-butyl-para-hydroxytoluene or flavonoid derivatives, such as alpha-glucosylrutin.

For the purposes of the present specification, photoprotective filters filter at least some of the light out of an incident ray of light by absorption or reflection.

Fatty and/or wax components to be used advantageously according to the invention can be chosen from the group of vegetable waxes, animal waxes, mineral waxes and petrochemical waxes. For example, candelilla wax, carnauba wax, Japan wax, esparto grass wax, cork wax, guaruma wax, rice germ oil wax, sugarcane wax, berry wax, ouricury wax, montan wax, jojoba wax, shea butter, beeswax, shellac wax, spermaceti, lanolin (wool wax), uropygial grease, ceresine, ozokerite (earth wax), paraffin waxes and microcrystalline waxes are favourable according to the invention provided the conditions required in the main claim are observed.

Further advantageous fatty and/or wax components are chemically modified waxes and synthetic waxes, such as, for example, those available under the trade names Syncrowax HRC (glyceryl tribehenate), and Syncrowax AW 1C (C₁₈₋₃₆-fatty acid) from CRODA GmbH, and montan ester waxes, sasol waxes, hydrogenated jojoba waxes, synthetic or modified beeswaxes (e.g. dimethicone copolyol beeswax and/or C₃₀₋₅₀-alkyl beeswax), polyalkylene waxes, polyethylene glycol waxes, but also chemically modified fats, such as, for example, hydrogenated plant oils (for example hydrogenated castor and/or hydrogenated coconut fatty glycerides), triglycerides, such as, for example, trihydroxysterin, fatty acids, fatty acid esters and glycol esters, such as, for example, C₂₀₋₄₀-alkyl stearate, C₂₀₋₄₀-alkylhydroxysteroyl stearate and/or glycol montanate. Also advantageous are certain organosilicon compounds which have similar physical properties to said fat and/or wax components, such as, for example, stearoxytrimethylsilane, provided the conditions required in the main claim are observed.

According to the invention, the fat and/or wax components can either be present individually or as a mixture. Any mixtures of such oil and wax components can also be used advantageously for the purposes of the present invention.

It is also advantageous for the purposes of the present invention to create cosmetic and dermatological preparations whose main purpose is not protection against sunlight, but which nevertheless contain UV protection substances. Thus, for example, UV-A and/or UV-B filter substances are usually incorporated into day creams or makeup products. UV protection substances, like antioxidants and, if desired, preservatives, also represent effective protection of the preparations themselves against spoilage. Also favourable are cosmetic and dermatological preparations which are present in the form of a sunscreen composition.

Accordingly, the preparations for the purposes of the present invention preferably comprise at least one UV-A and/or UV-B filter substance. The formulations may, but do not necessarily, optionally also comprise one or more organic and/or inorganic pigments as UV filter substances, which may be present in the water phase and/or the oil phase.

Preferred inorganic pigments are metal oxides and/or other metal compounds which are sparingly soluble or insoluble in water, in particular oxides of titanium (TiO₂), zinc (ZnO), iron (e.g. Fe₂O₃), zirconium (ZrO₂), silicon (SiO₂), manganese (e.g. MnO), aluminium (Al₂O₃), cerium (e.g. Ce₂O₃), mixed oxides of the corresponding metals, and mixtures of such oxides, and the sulphate of barium (BaSO₄).

The titanium dioxide pigments may be present either in the crystal modification rutile, or else in the form of anatase and may, for the purposes of the present invention, be advantageously surface-treated (“coated”), the intention being to form or retain, for example, a hydrophilic, amphiphilic or hydrophobic character. This surface treatment can involve providing the pigments with a thin hydrophilic and/or hydrophobic inorganic and/or organic layer by processes known per se. The various surface coatings can also comprise water for the purposes of the present invention.

Described coated and uncoated titanium dioxides can also be used for the purposes of the present invention in the form of commercially available oily or aqueous predispersions. Dispersion auxiliaries and/or solubilization promoters may advantageously be added to these predispersions.

The titanium dioxides according to the invention are characterized by a primary particle size between 10 nm to 150 nm.

| | | Additional constituents | |
|---|---|---|---|
| Trade name | Coating | of the predispersion | Manufacturer |
| MT-100TV | Aluminium hydroxide | — | Tayca Corporation |
| | stearic acid | | |
| MT-100Z | Aluminium hydroxide | — | Tayca Corporation |
| | stearic acid | | |
| MT-100F | Stearic acid | — | Tayca Corporation |
| | iron oxide | | |
| MT-500SAS | Alumina, silica | — | Tayca Corporation |
| | silicone | | |
| MT-100AQ | Silica | — | Tayca Corporation |
| | aluminium hydroxide | | |
| | alginic acid | | |
| Eusolex T-2000 | Alumina | — | Merck KGaA |
| | simethicones | | |
| Eusolex TS | Alumina, stearic acid | — | Merck KGaA |
| Titanium dioxide | None | — | Degussa |
| P25 | | | |
| Titanium dioxide | Octyltrimethylsilane | — | Degussa |
| T805 | | | |
| (Uvinul TiO₂) | | | |
| UV-Titan X170 | Alumina | — | Kemira |
| | dimethicones | | |
| UV-Titan X161 | Alumina, silica | — | Kemira |
| | stearic acid | | |
| Tioveil AQ 10PG | Alumina | Water | Solaveil |
| | silica | propylene glycol | Uniquema |
| Mirasun TiW 60 | Alumina | Water | Rhone-Poulenc |
| | silica | | |

For the purposes of the present invention, particularly preferred titanium dioxides are MT-100Z and MT-100TV from Tayca Corporation, Eusolex T-2000 and Eusolex TS from Merck and Titanium dioxide T805 from Degussa.

For the purposes of the present invention, zinc oxides can also be used in the form of commercially available oily or aqueous predispersions. Zinc oxide particles suitable according to the invention and predispersions of zinc oxide particles are characterized by a primary particle size of <300 nm and are available under the following trade names from the companies listed:

| Trade name | Coating | Manufacturer |
|---|---|---|
| Z-Cote HP1 | 2% Dimethicones | BASF |
| Z-Cote | / | BASF |
| ZnO NDM | 5% Dimethicones | H&R |
| MZ 707M | 7% Dimethicones | M. Tayca Corp. |
| Nanox 500 | / | Elementis |
| ZnO Neutral | / | H&R |

Particularly preferred zinc oxides for the purposes of the invention are Z-Cote HP1 from BASF and zinc oxide NDM from Haarmann & Reimer.

The total amount of one or more inorganic pigments in the finished cosmetic preparation is advantageously chosen from the range 0.1% by weight to 25% by weight, preferably 0.5% by weight to 18% by weight.

An advantageous organic pigment for the purposes of the present invention is 2,2′-methylenebis(6-(2H-benzotriazol-2-yl )-4-(1,1,3,3-tetramethylbutyl)phenol) [INCI:Bisoctyltriazole], which is characterized by the chemical structural formula 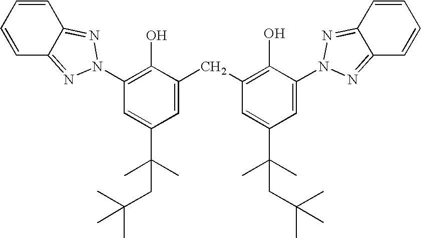
and is available under the trade name Tinosorb® M from CIBA-Chemikalien GmbH.

Advantageous UV-A filter substances for the purposes of the present invention are dibenzoylmethane derivatives, in particular 4-(tert-butyl)-4′-methoxydibenzoylmethane (CAS No. 70356-09-1), which is sold by Givaudan under the name Parsole® 1789 and by Merck under the trade name Eusolex® 9020.

Further advantageous UV-A filter substances are phenylene-1,4-bis(2-benzimidazyl)-3,3′-5,5′-tetrasulphonic acid 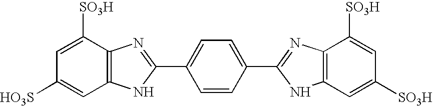
and its salts, particularly the corresponding sodium, potassium or triethanolammonium salts, in particular phenylene-1,4-bis(2-benzimidazyl)-3,3′-5,5′-tetrasulphonic bis-sodium salt 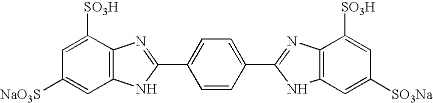
with the INCI name Bisimidazylate, which is available, for example, under the trade name Neo Heliopan AP from Haarmann & Reimer.

Also advantageous are 1,4-di(2-oxo-10-sulpho-3-bornylidenemethyl)benzene and salts thereof (in particular the corresponding 10-sulphato compounds, in particular the corresponding sodium, potassium or triethanolammonium salt), which is also referred to as benzene-1,4-di(2-oxo-3-bornylidenemethyl-10-sulphonic acid) and is characterized by the following structure:

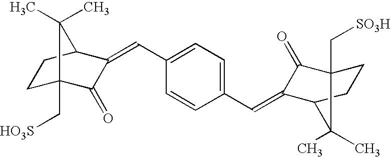

Further advantageous UV-A filter substances are hydroxybenzophenones which are characterized by the following structural formula: 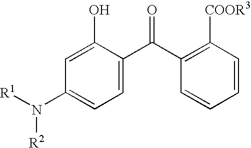
in which
R¹ and R², independently of one another, are hydrogen, C₁-C₂₀-alkyl, C₃-C₁₀-cycloalkyl or C₃-C₁₀-cycloalkenyl, where the substituents R¹ and R², together with the nitrogen atom to which they are bonded, can form a 5-membered or 6-membered ring and
R³ is a C₁-C₂₀-alkyl radical.

A particularly advantageous hydroxybenzophenone for the purposes of the present invention is hexyl 2-(4′-diethylamino-2′-hydroxybenzoyl)benzoate (also: aminobenzophenone), which is characterized by the following structure: 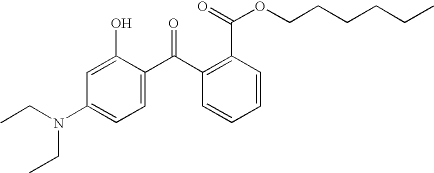
and is available under the trade name Uvinul A Plus from BASF.

Advantageous UV filter substances for the purposes of the present invention are also so-called broadband filters, i.e. filter substances which absorb both UV-A and also UV-B radiation.

Advantageous broadband filters or UV-B filter substances are, for example, bisresorcinyltriazine derivatives having the following structure: 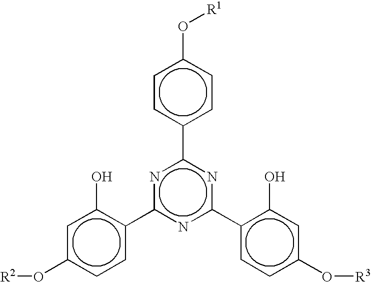
where R¹, R² and R³, independently of one another, are chosen from the group of branched and unbranched alkyl groups having 1 to 10 carbon atoms, or are a single hydrogen atom. Particular preference is given to 2,4-bis{[4-(2-ethylhexyloxy)-2-hydroxy]phenyl}-6-(4-methoxyphenyl)-1,3,5-triazine (INCI: Aniso Triazine), which is available under the trade name Tinosorb® S from CIBA-Chemikalien GmbH.

For the purposes of the present invention, particularly advantageous preparations which are characterized by high or very high UV-A protection preferably comprise two or more UV-A and/or broadband filters, in particular dibenzoylmethane derivatives [for example 4-(tert-butyl)-4′-methoxydibenzoylmethane], benzotriazole derivatives [for example 2,2′-methylenebis(6-(2H-benzotriazol-2-yl)-4-(1,1,3,3-tetramethylbutyl)phenol)], phenylene-1,4-bis(2-benzimidazyl)-3,3′-5,5′-tetrasulphonic acid and/or its salts, 1,4-di(2-oxo-10-sulpho-3-bornylidenemethyl)benzene and/or salts thereof and/or 2,4-bis{[4-(2-ethylhexyloxy)-2-hydroxy]phenyl}-6-(4-methoxyphenyl)-1,3,5-triazine, in each case individually or in any combinations with one another.

Other UV filter substances, which have the structural formula 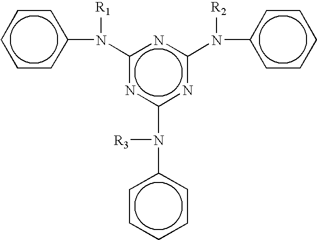
are also advantageous UV filter substances for the purposes of the present invention, for example the s-triazine derivatives described in European laid-open specification EP 570 838 A1, whose chemical structure is expressed by the generic formula 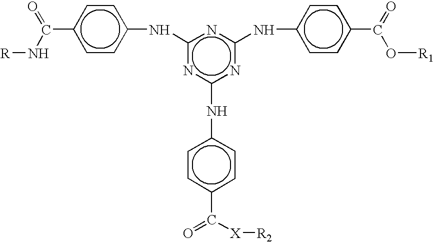
where
R is a branched or unbranched C₁-C₁₈-alkyl radical, a C₅-C₁₂-cycloalkyl radical, optionally substituted by one or more C₁-C₄-alkyl groups,
X is an oxygen atom or an NH group,
R₁ is a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula 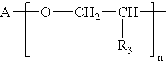
   in which
   A is a branched or unbranched C₁-C₁₈-alkyl radical, a C₅-C₁₂-cycloalkyl or aryl radical, optionally substituted by one or more C₁-C₄-alkyl groups,
   R₃ is a hydrogen atom or a methyl group,
   n is a number from 1 to 10,
R₂ is a branched or unbranched C₁-C₁₈-alkyl radical, a C₅-C₁₂-cycloalkyl radical, optionally substituted by one or more C₁-C₄-alkyl groups, when X is the NH group, and
   a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula 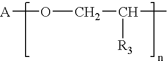
      in which
   A is a branched or unbranched C₁-C₁₈-alkyl radical, a C₅-C₁₂-cycloalkyl or aryl radical, optionally substituted by one or more C₁-C₄-alkyl groups,
   R₃ is a hydrogen atom or a methyl group,
   n is a number from 1 to 10,
   when X is an oxygen atom.

A particularly preferred UV filter substance for the purposes of the present invention is also an unsymmetrically substituted s-triazine, the chemical structure of which is expressed by the formula 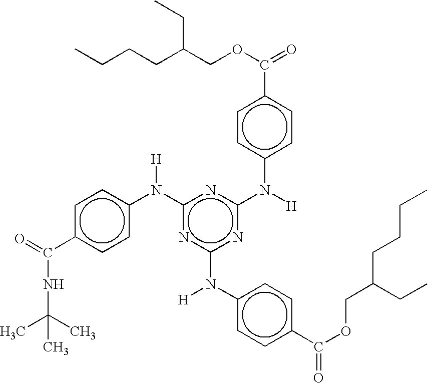
and which is also referred to below as dioctylbutylamidotriazone (INCI: Dioctylbutamidotriazone), and is available under the trade name UVASORB HEB from Sigma 3V.

Also advantageous for the purposes of the present invention is a symmetrically substituted s-triazine, tris(2-ethylhexyl) 4,4′,4″-(1,3,5-triazine-2,4,6-triyltriimino)trisbenzoate, synonym: 2,4,6-tris[anilino-(p-carbo-2′-ethyl-1′-hexyloxy)]-1,3,5-triazine (INCI: Octyl Triazone), which is marketed by BASF Aktiengesellschaft under the trade name UVINUL® T 150.

European laid-open specification 775 698 also describes preferred bisresorcinyltriazine derivatives to be used, the chemical structure of which is expressed by the generic formula 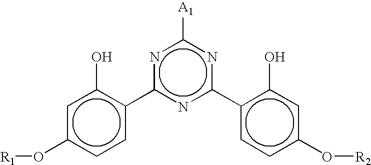
where R₁, R₂ and A₁ represent very different organic radicals.

Also advantageous for the purposes of the present invention are 2,4-bis{[4-(3-sulphonato)-2-hydroxypropyloxy)-2-hydroxy]phenyl}-6-(4-methoxyphenyl)-1,3,5-triazine sodium salt, 2,4-bis{[4-(3-(2-propyloxy)-2-hydroxypropyloxy)-2-hydroxy]phenyl}-6-(4-methoxyphenyl)-1,3,5-triazine, 2,4-bis{[4-(2-ethylhexyloxy)-2-hydroxy]phenyl}-6-[4-(2-methoxyethylcarboxyl)phenylamino]-1, 3, 5-triazine, 2,4-bis{[4-(3-(2-propyloxy)-2-hydroxypropyloxy)-2-hydroxy]phenyl}-6-[4-(2-ethylcarboxyl)phenylamino]-1,3,5-triazine, 2,4-bis{[4-(2-ethylhexyloxy)-2-hydroxy]phenyl}-6-(1-methylpyrrol-2-yl)-1,3,5-triazine, 2,4-bis{[4-tris(trimethyl-siloxysilylpropyloxy)-2-hydroxy]phenyl}-6-(4-methoxyphenyl)-1,3,5-triazine, 2,4-bis{[4-(2″-methylpropenyloxy)-2-hydroxy]phenyl}-6-(4-methoxyphenyl)-1,3,5-triazine and 2,4-bis{[4-(1′,1′,1′,3′,5′,5′,5′-heptamethylsiloxy-2″-methylpropyloxy)-2-hydroxy]phenyl}-6-(4-methoxyphenyl)-1,3,5-triazine.

An advantageous broadband filter for the purposes of the present invention is 2,2′-methylenebis(6-(2H-benzotriazol-2-yl)-4-(1,1,3,3-tetramethylbutyl)phenol), which is characterized by the chemical structural formula 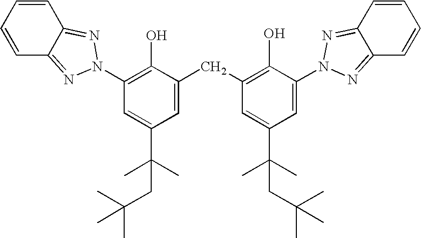
and is available under the trade name Tinosorb®) M from CIBA-Chemikalien GmbH.

Another advantageous broadband filter for the purposes of the present invention is 2-(2H-benzotriazol-2-yl)-4-methyl-6-[2-methyl-3-[1,3,3,3-tetramethyl-1-[(trimethylsilyl)oxy]disiloxanyl]propyl]phenol (CAS No.: 155633-54-8) having the INCI name Drometrizole Trisiloxane, which is characterized by the chemical structural formula

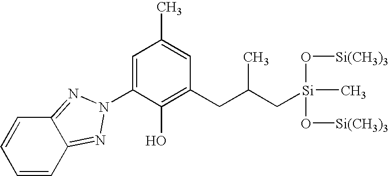

The UV-B and/or broadband filters can be oil-soluble or water-soluble. Examples of advantageous oil-soluble UV-B and/or broadband filter substances are:
3-benzylidenecamphor derivatives, preferably 3-(4-methylbenzylidene)camphor, 3-benzylidenecamphor;
4-aminobenzoic acid derivatives, preferably 2-ethylhexyl 4-(dimethylamino)-benzoate, amyl 4-(dimethylamino)benzoate;
2,4,6-trianilino(p-carbo-2′-ethyl-1′-hexyloxy)-1,3,5-triazine;
esters of benzalmalonic acid, preferably di(2-ethylhexyl) 4-methoxybenzalmalonate,
esters of cinnamic acid, preferably 2-ethylhexyl 4-methoxycinnamate, isopentyl 4-methoxycinnamate;
derivatives of benzophenone, preferably 2-hydroxy-4-methoxybenzophenone, 2-hydroxy-4-methoxy-4′-methylbenzophenone, 2,2′-d ihydroxy-4-methoxybenzo-phenone
and UV filters bonded to polymers.

Examples of advantageous water-soluble UV-B and/or broadband filter substances are:
salts of 2-phenylbenzimidazole-5-sulphonic acid, such as its sodium, potassium or its triethanolammonium salt, and also the sulphonic acid itself;
sulphonic acid derivatives of 3-benzylidenecamphor, such as, for example, 4-(2-oxo-3-bornylidenemethyl)benzenesulphonic acid, 2-methyl-5-(2-oxo-3-bornylidenemethyl)sulphonic acid and salts thereof.

Particularly advantageous UV filter substances which are liquid at room temperature for the purposes of the present invention are homomenthyl salicylate (INCI: Homosalate), 2-ethylhexyl 2-hydroxybenzoate (2-ethylhexyl salicylate, INCI: Octyl Salicylate), 4-isopropylbenzyl salicylate and esters of cinnamic acid, preferably (2-ethylhexyl) 4-methoxycinnamate (INCI: Octyl Methoxycinnamate) and isopentyl 4-methoxycinnamate (INCI: Isoamyl p-Methoxycinnamate), 3-(4-(2,2-bisethoxycarbonylvinyl )phenoxy)propenyl)methoxysiloxane/dimethylsiloxane copolymer (INCI: Dimethicodiethylbenzalmalonate), which is available, for example, under the trade name Parsol® SLX from Hoffmann La Roche.

A further photoprotective filter substance which can be used advantageously according to the invention is ethylhexyl 2-cyano-3,3-diphenylacrylate (octocrylene), which is available from BASF under the name Uvinul® N 539 and is characterized by the following structure:

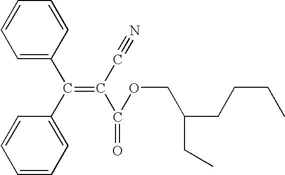

It can also be of considerable advantage to use polymer-bonded or polymeric UV filter substances in the preparations according to the present invention, in particular those described in WO-A-92/20690.

The list of specified UV filters which can be used for the purposes of the present invention is not of course intended to be limiting.

The preparations according to the invention advantageously comprise the substances which absorb UV radiation in the UV-A and/or UV-B region in a total amount of, for example, 0.1% by weight to 30% by weight, preferably 0.5 to 20% by weight, in particular 1.0 to 15.0% by weight, in each case based on the total weight of the preparations, in order to provide cosmetic preparations which protect the hair or the skin from the entire range of ultraviolet radiation. They can also be used as sunscreens for the hair or the skin.

The preparations according to the invention advantageously additionally comprise active ingredients which positively influence the condition of the skin. For example, it has been found that active ingredients for positively influencing ageing skin, which decrease the formation of wrinkles or else existing wrinkles (bioquinones, in particular ubiquinone Q10) or promote the restructuring of the connective tissue (isoflavone) can be used very readily in the formulations according to the invention. It has also been found that the formulations are particularly suitable for using active ingredients for supporting functions of the skin in (ageing) dry skin (serinol, osmolytes such as taurine, sodium chloride, sea salt etc.). In a similar way, the incorporation of active ingredients for alleviating and/or positively influencing irritative skin conditions, whether in the case of sensitive skin in general or in the case of skin irritated by noxae (UV light, chemicals), has proven to be advantageous. Active ingredients which can be mentioned here are, for example, sericosides, ursolic acid, glycyrrhizinic acid, dexpanthenol, inhibitors of prostaglandin metabolism (in particular of cyclooxygenase) and of leucotriene metabolism (in particular of 5-lipoxygenase), such as, for example, acetylsalicylic acid and hamamelis ingredients. The incorporation of modulators of pigmentation has also proven to be advantageous. Active ingredients to be mentioned here are those which reduce pigmentation in the skin and thus lead to a cosmetically desired lightening of the skin and/or reduce the appearance of age spots and/or lighten existing age spots (for example tyrosine sulphate, dioic acid [8-hexadecene-1,16-dicarboxylic acid], lipoic acid and liponamide, extracts of liquorice, kojic acid, hydroquinone, arbutin, fruit acids, in particular alphahydroxy acids (AHAs), bearberry (Uvae ursi), ursolic acid, ascorbic acid, green tea extracts). In a similar manner, the formulations according to the invention have proven to be an excellent base for active ingredients which bring about an increased or more rapid tanning of the skin (Advanced Glycation Endproducts (AGE), lipofuscins, nucleic acid oligonucleotides, purines and pyrimidines, NO-releasing substances), be it with or without the influence of UV light.

In addition, antioxidants are advantageously chosen from the group consisting of amino acids (e.g. glycine, lysine, arginine, cysteine, histidine, tyrosine, tryptophan) and derivatives thereof (as salt, ester, ether, sugar, nucleotide, nucleoside, peptide and lipid compound), imidazoles (e.g. urocanic acid) and derivatives thereof (as salt, ester, ether, sugar, nucleotide, nucleoside, peptide and/or lipid compound), peptides, such as D,L-carnosine, D-carnosine, L-carnosine, anserine and derivatives thereof (as salt, ester, ether, sugar, thiol, nucleotide, nucleoside, peptide and lipid compound), carotenoids, carotenes (e.g. α-carotene, β-carotene, ψ-lycopene, phytoene) and derivatives thereof (as salt, ester, ether, sugar, nucleotide, nucleoside, peptide and/or lipid compound), chlorogenic acid and derivatives thereof (as salt, ester, ether, sugar, thiol, nucleotide, nucleoside, peptide and/or lipid compound), aurothioglucose, propylthiouracil and other thiols (e.g. thioredoxin, lipoic acid, glutathione, cysteine, cystine, cystamine and their glycosyl, N-acetyl, methyl, ethyl, propyl, amyl, butyl and lauryl, palmitoyl, oleyl, γ-linoleyl, cholesteryl and glyceryl esters) and salts thereof, dilauryl thiodipropionate, distearyl thiodipropionate, thiodipropionic acid and derivatives thereof (as salt, ester, ether, sugar, thiol, nucleotide, nucleoside, peptide and/or lipid compound), and sulphoximine compounds (e.g. homocysteine sulphoximine, buthionine sulphones, penta-, hexa-, heptathionine sulphoximine) in very low tolerated doses (e.g. pmol to μmol/kg). Also (metal) chelating agents (e.g. apoferritin, desferral, lactoferrin, α-hydroxy fatty acids, palmitic acid, phytic acid) and derivatives thereof (as salt, ester, ether, sugar, thiol, nucleotide, nucleoside, peptide and/or lipid compound), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, furfurylidenesorbitol and derivatives thereof, ubiquinone, ubiquinol, plastoquinone and derivatives thereof (as salt, ester, ether, sugar, thiol, nucleotide, nucleoside, peptide and lipid compound), vitamin C and derivatives (e.g. ascorbyl palmitate, Mg ascorbyl phosphate, ascorbyl acetate), tocopherols and derivatives (e.g. vitamin E acetate), and phenolic compounds and plant extracts comprising these, such as, for example, flavonoids (e.g. glycosylrutin, ferulic acid, caffeic acid), furfurylideneglucitol, butylhydroxytoluene, butylhydroxyanisole, nordihydroguaiacic acid, nordihydroguaiaretic acid, trihydroxybutyrophenone and derivatives thereof (as salt, ester, ether, sugar, nucleotide, nucleoside, peptide and lipid compound). Uric acid and derivatives thereof, mannose and derivatives thereof (as salt, ester, ether, sugar, thiol, nucleotide, nucleoside, peptide and lipid compound). Zinc and derivatives thereof (e.g. ZnO, ZnSO₄), selenium and derivatives thereof (e.g. selenomethionine, ebselen), stilbenes and derivatives thereof (e.g. stilbene oxide, trans-stilbene oxide) and the derivatives suitable according to the invention (as salt, ester, ether, sugar, thiol, nucleotide, nucleoside, peptide and/or lipid compound) of these active ingredients mentioned.

Emulsifiers according to the invention can advantageously be chosen from the group of nonionic, anionic, cationic or amphoteric emulsifiers.

The nonionic emulsifiers include

a) partial fatty acid esters and fatty acid esters of polyhydric alcohols and ethoxylated derivatives thereof (e.g. glyceryl monostearate, sorbitan stearate, glyceryl stearyl citrate, sucrose stearate)
b) ethoxylated fatty alcohols and fatty acids
c) ethoxylated fatty amines, fatty acid amides, fatty acid alkanolamides
d) alkylphenol polyglycol ethers (e.g. Triton X)
   The anionic emulsifiers include
a) soaps (e.g. sodium stearate)
b) fatty alcohol sulphates
c) mono-, di- and trialkyl phosphoric esters and ethoxylates thereof
   The cationic emulsifiers include
a) quaternary ammonium compounds with a long-chain aliphatic radical, e.g. distearyldimonium chloride
   The amphoteric emulsifiers include
a) alkylaminoalkanecarboxylic acids
b) betaines, sulphobetaines
c) imidazoline derivatives
   There are also naturally occurring emulsifiers, which include beeswax, wool wax, lecithin and sterols.

O/W emulsifiers can, for example, advantageously be chosen from the group of polyethoxylated or polypropoxylated or polyethoxylated and polypropoxylated products, e.g.:
of fatty alcohol ethoxylates
of ethoxylated wool wax alcohols,
of polyethylene glycol ethers of the general formula R—O—(—CH₂—CH₂—O—)ₙ—R′,
of fatty acid ethoxylates of the general formula
   R—COO—(CH₂—CH₂—O—)ₙ—H,
of etherified fatty acid ethoxylates of the general formula
   R—COO—(CH₂—CH₂—O—)ₙ—R′,
of esterified fatty acid ethoxylates of the general formula
   R—COO—(CH₂—CH₂—O—)ₙ—C(O)—R′,
of polyethylene glycol glycerol fatty acid esters
of ethoxylated sorbitan esters
of cholesterol ethoxylates
of ethoxylated triglycerides
of alkyl ether carboxylic acids of the general formula R—O—(—CH₂—CH₂—O—)ₙ—CH₂—COOH and n is a number from 5 to 30,
of polyoxyethylene sorbitol fatty acid esters,
of alkyl ether sulphates of the general formula R—O—(—CH₂—CH₂—O—)ₙ—SO₃—H
of fatty alcohol propoxylates of the general formula
   R—O—(CH₂—CH(CH₃)—O—)ₙ—H,
of polypropylene glycol ethers of the general formula
   R—O—(CH₂—CH(CH₃)—O—)ₙ—R′,
of propoxylated wool wax alcohols,
of etherified fatty acid propoxylates R—COO—(—CH₂—CH(CH₃)—O—)ₙ—R′,
of esterified fatty acid propoxylates of the general formula
   R—COO—(—CH₂—CH(CH₃)—O—)ₙ—C(O)—R′,
of fatty acid propoxylates of the general formula
   R—COO—(CH₂—CH(CH₃)—O—)ₙ—H,
of polypropylene glycol glycerol fatty acid esters
of propoxylated sorbitan esters
of cholesterol propoxylates
of propoxylated triglycerides
of alkyl ether carboxylic acids of the general formula
   R—O—(CH₂—CH(CH₃)—O—)ₙ—CH₂—COOH
of alkyl ether sulphates and the acids on which these sulphates are based of the general formula R—O—(CH₂—CH(CH₃)—O—)ₙ—SO₃—H
of fatty alcohol ethoxylates/propoxylates of the general formula
   R—O—Xₙ—Yₘ—H,
of polypropylene glycol ethers of the general formula
   R—O—Xₙ—Yₘ—R′,
of etherified fatty acid propoxylates of the general formula
   R—COO—Xₙ—Yₘ—R′,
of fatty acid ethoxylates/propoxylates of the general formula
   R—COO—Xₙ—Yₘ—H.

According to the invention, the polyethoxylated or polypropoxylated or polyethoxylated and polypropoxylated O/W emulsifiers are particularly advantageously chosen from the group of substances with HLB values of 11-18, very particularly advantageously with HLB values of 14.5-15.5, if the O/W emulsifiers have saturated radicals R and R′. If the O/W emulsifiers have unsaturated radicals R and/or R′, or if isoalkyl derivatives are present, then the preferred HLB value of such emulsifiers may also be lower or greater.

It is advantageous to choose the fatty alcohol ethoxylates from the group of ethoxylated stearyl alcohols, cetyl alcohols, cetylstearyl alcohols (cetearyl alcohols). Particular preference is given to polyethylene glycol(13) stearyl ether (steareth-13), polyethylene glycol(14) stearyl ether (steareth-14), polyethylene glycol(15) stearyl ether (steareth-15), polyethylene glycol(16) stearyl ether (steareth-16), polyethylene glycol(17) stearyl ether (steareth-17), polyethylene glycol(18) stearyl ether (steareth-18), polyethylene glycol(19) stearyl ether (steareth-19), polyethylene glycol(20) stearyl ether (steareth-20), polyethylene glycol(12) isostearyl ether (isosteareth-12), polyethylene glycol(13) isostearyl ether (isosteareth-13), polyethylene glycol(14) isostearyl ether (isosteareth-14), polyethylene glycol(15) isostearyl ether (isosteareth-15), polyethylene glycol(16) isostearyl ether (isosteareth-16), polyethylene glycol(17) isostearyl ether (isosteareth-17), polyethylene glycol(18) isostearyl ether (isosteareth-18), polyethylene glycol(19) isostearyl ether (isosteareth-19), polyethylene glycol(20) isostearyl ether (isosteareth-20), polyethylene glycol(13) cetyl ether (ceteth-13), polyethylene glycol(14) cetyl ether (ceteth-14), polyethylene glycol(15) cetyl ether (ceteth-15), polyethylene glycol(16) cetyl ether (ceteth-16), polyethylene glycol(17) cetyl ether (ceteth-17), polyethylene glycol(18) cetyl ether (ceteth-18), polyethylene glycol(19) cetyl ether (ceteth-19), polyethylene glycol(20) cetyl ether (ceteth-20), polyethylene glycol(13) isocetyl ether (isoceteth-13), polyethylene glycol(14) isocetyl ether (isoceteth-14), polyethylene glycol(15) isocetyl ether (isoceteth-15), polyethylene glycol(16) isocetyl ether (isoceteth-16), polyethylene glycol(17) isocetyl ether (isoceteth-17), polyethylene glycol(18) isocetyl ether (isoceteth-18), polyethylene glycol(19) isocetyl ether (isoceteth-19), polyethylene glycol(20) isocetyl ether (isoceteth-20), polyethylene glycol(12) oleyl ether (oleth-12), polyethylene glycol(13) oleyl ether (oleth-13), polyethylene glycol(14) oleyl ether (oleth-14), polyethylene glycol(15) oleyl ether (oleth-15), polyethylene glycol(12) lauryl ether (laureth-12), polyethylene glycol(12) isolauryl ether (isolaureth-12), polyethylene glycol(13) cetylstearyl ether (ceteareth-13), polyethylene glycol(14) cetylstearyl ether (ceteareth-14), polyethylene glycol(15) cetylstearyl ether (ceteareth-15), polyethylene glycol(16) cetylstearyl ether (ceteareth-16), polyethylene glycol(17) cetylstearyl ether (ceteareth-17), polyethylene glycol(18) cetylstearyl ether (ceteareth-18), polyethylene glycol(19) cetylstearyl ether (ceteareth-19), and polyethylene glycol(20) cetylstearyl ether (ceteareth-20).

It is also advantageous to choose the fatty acid ethoxylates from the following group: polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, and polyethylene glycol(20) oleate.

Ethoxylated alkyl ether carboxylic acid or salts thereof which may be used advantageously is sodium laureth-11 carboxylate.

As alkyl ether sulphate, sodium laureth-14 sulphate can be used advantageously.

As ethoxylated cholesterol derivative, polyethylene glycol(30) cholesteryl ether can be used advantageously. Polyethylene glycol(25) soyasterol has also proven useful.

Ethoxylated triglycerides which can be used advantageously are the polyethylene glycol(60) evening primrose glycerides.

It is also advantageous to choose the polyethylene glycol glycerol fatty acid esters from the group consisting of polyethylene glycol(20) glyceryl laurate, polyethylene glycol(21) glyceryl laurate, polyethylene glycol(22) glyceryl laurate, polyethylene glycol(23) glyceryl laurate, polyethylene glycol(6) glyceryl caprate/caprinate, polyethylene glycol(20) glyceryl oleate, polyethylene glycol(20) glyceryl isostearate, polyethylene glycol(18) glyceryl oleate/cocoate.

It is likewise favourable to choose the sorbitan esters from the group consisting of polyethylene glycol(20) sorbitan monolaurate, polyethylene glycol(20) sorbitan monostearate, polyethylene glycol(20) sorbitan monoisostearate, polyethylene glycol(20) sorbitan monopalmitate, polyethylene glycol(20) sorbitan monooleate.

Advantageous W/O emulsifiers which may be used are: fatty alcohols having 8 to 30 carbon atoms, monoglycerol esters of saturated and/or unsaturated, branched and/or unbranched alkanecarboxylic acids of chain length from 8 to 24, in particular 12-18, carbon atoms, diglycerol esters of saturated and/or unsaturated, branched and/or unbranched alkanecarboxylic acids of chain length from 8 to 24, in particular 12-18, carbon atoms, monoglycerol ethers of saturated and/or unsaturated, branched and/or unbranched alcohols of chain length from 8 to 24, in particular 12-18, carbon atoms, diglycerol ethers of saturated and/or unsaturated, branched and/or unbranched alcohols of chain length from 8 to 24, in particular 12-18, carbon atoms, propylene glycol esters of saturated and/or unsaturated, branched and/or unbranched alkanecarboxylic acids of chain length from 8 to 24, in particular 12-18, carbon atoms, and sorbitan esters of saturated and/or unsaturated, branched and/or unbranched alkanecarboxylic acids of chain length from 8 to 24, in particular 12-18, carbon atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol(2) stearyl ether (steareth-2), glyceryl monolaurate, glyceryl monocaprinate, glyceryl monocaprylate.

Polymers which serve as gel formers or viscosity-increasing substances can optionally be used advantageously for the preparations according to the invention. Examples thereof are, inter alia, carbopols, i.e. polymers of acrylic acid, in particular also acrylate-alkyl acrylate copolymers. Advantageous carbopols are, for example, the grades 980, 981, 984, 1342, 1382, 2984 and 5984, likewise the ETD grades 2001, 2020, 2050 and Carbopol Ultrez 10, Ultrez 21, PVM/MA decadiene crosspolymer (trade name Stabileze 06), polyglyceryl methacrylate, and polyacrylamide. Also advantageous are the thickener grades Sepigel 305, Sepigel 501, Sepigel 502, Simulgel 600, Simulgel A, Simulgel EG, Simulgel EG-SL and Simulgel NT (all from Seppic). Moreover, further gel formers based on AMPS or AMPS copolymer may be present, such as, for example, Hostacerin AMPS (AMPS polymer), Aristoflex AVC (AMPS-VP copolymer), Aristoflex HMB.

Further advantageous thickening polymers for the preparations according to the invention are xanthan gum, polyvinylpyrrolidone, cellulose derivatives, in particular cellulose ethers, such as, for example, hydroxypropylmethylcellulose, starch and starch derivatives, hyaluronic acid, and carob seed flour.

For all this it is possible in individual cases that the above-stated concentration data is easily exceeded or not reached and nevertheless preparations according to the invention are obtained. In view of the widely scattered diversity of suitable components of such preparations, this comes as no surprise to the person skilled in the art, meaning that he knows that such excesses or shortfalls do not depart from the essence of the present invention.

The examples below are intended to illustrate the present invention without limiting it. The numerical values in the examples are percentages by weight, based on the total weight of the particular preparations.

### EXAMPLES

Particles according to the invention can be prepared in two ways, namely by means of the vaporization method and by means of the emulsion method.

In the evaporation method, the wax to be used is firstly dissolved in a volatile organic solvent, and then a polyol-in-solvent emulsion is prepared from this organic phase and an active ingredient-containing polyol phase. This can take place by the customary methods for the preparation of an emulsion (high-pressure homogenizers, ultrasound devices, rotor-stator devices). From this preemulsion and a water phase is then prepared a polyol-in-solvent-in-water emulsion, from which the solvent is removed by applying a subatmospheric pressure and/or by heating. The contained wax becomes gradually solid during the process, as a result of which the emulsion converts to a polyol-in-wax-in-water suspension. Table 1 shows exemplary compositions.

TABLE 1
Examples of the preparation of particles according
to the invention by the vaporization method

| Phase | Chemical name | Example 1 | Example 2 |
|---|---|---|---|
| Polyol phase | Butylene glycol | — | 20 |
| | Glycerol | 16 | — |
| | Ascorbic acid | 1 | — |
| | Genistein | — | 0.25 |
| | Sodium chloride | 0.5 | 0.5 |
| Solvent/wax | n-Hexane | 30 | 30 |
| phase | Ceresine | 30 | 30 |
| | PEG-30 | 3 | 3 |
| | dipolyhydroxystearate | | |
| | BHT | 0.1 | 0.1 |
| Water phase | Glyceryl stearate citrate | 4 | — |
| | Polyglyceryl-3 | — | 5 |
| | methylglucose distearate | | |
| | Phenonip | 0.5 | 0.5 |
| | NaOH | Qs | Qs |
| | Perfume | Qs | Qs |
| | Deionized water | ad 100 | ad 100 |

In the emulsion method, a polyol-in-wax preemulsion is firstly prepared above the melting temperature of the wax from an active ingredient-containing polyol phase and a wax phase, for which purpose it is possible to use the customary methods for the preparation of an emulsion which are already mentioned above.

From this preemulsion and a water phase is prepared, still above the melting temperature of the wax phase, a polyol-in-wax-in-water emulsion from which a polyol-in-wax-in-water suspension is prepared by cooling to below the melting temperature of the wax phase.

TABLE 2
Examples of possible compositions of wax phases
(data in weight percent based on the total weight
of the polyol-in-wax preemulsion)

| Chemical name | L1 | L2 | L3 | L4 | L5 | L6 | L7 |
|---|---|---|---|---|---|---|---|
| Ceresine | 44.5 | — | — | 20 | 10 | — | 10 |
| Stearyl stearate | — | 30 | — | 25 | 29.5 | 10 | 10 |
| Hydroxyocta- | 5 | — | — | 3 | | 3 | 3 |
| cosanyl hydroxy- | | | | | | | |
| stearate | | | | | | | |
| Tribehenin | — | — | 49.5 | — | 10 | 20 | 10 |
| Eucerit | — | 5 | — | — | 5 | 4.5 | 2 |
| PEG-30 dipoly- | — | — | 5 | 4 | — | — | 2 |
| hydroxystearate | | | | | | | |
| TiO₂ | 10 | — | — | 8 | | 7 | 6.5 |
| Lischu S | — | 5 | 5 | — | 5 | 5 | 6 |
| BHT | 0.5 | — | 0.5 | — | 0.5 | 0.5 | 0.5 |
| Total wax phase | 60 | 40 | 60 | 60 | 60 | 50 | 50 |

TABLE 3
Polyol phases (data in weight percent based
on the total weight of the polyol phase)

| Chemical name | P1 | P2 | P3 |
|---|---|---|---|
| Glycerol | 96 | — | 46.4 |
| Butylene glycol | — | 93 | 46.3 |
| Sodium chloride | 0.3 | 0.3 | 0.3 |
| EDTA disodium salt | 0.3 | — | 0.3 |
| Ascorbic acid | 3.4 | — | — |
| Genistein | — | 6.7 | — |
| Creatine | — | — | 6.7 |

TABLE 4
Water phases (data in weight percent based on the total
weight of the polyol-in-wax-in-water suspension)

| Chemical name | W1 | W2 | W3 | W4 |
|---|---|---|---|---|
| Deionized water | Ad 60 | ad 50 | ad 70 | ad 60 |
| Glycerol | 10 | 5 | 12 | 10 |
| PEG-40 stearate | 1 | — | — | 1 |
| Glyceryl stearate | 2.5 | — | — | 4 |
| Polyglyceryl-3 | — | 4 | — | — |
| methylglucose distearate | | | | |
| Glyceryl stearate citrate | — | — | 2 | — |
| Phenonip | 0.4 | 0.4 | 0.4 | 0.4 |
| Carbomer | 0.1 | 0.2 | 0.2 | 0.1 |
| NaOH | Qs | qs | qs | Qs |
| Perfume | Qs | qs | qs | Qs |

Table 2 to Table 4 show compositions of the corresponding phases by way of example. Table 5 gives some of the active ingredient-containing particle suspensions prepared therefrom. The particular fractions of the phases are revealed by combining the wax phases listed in Table 2 with corresponding amounts of polyol phase (thus L1 with 40% polyol phase, L2 with 60% polyol phase etc.), in order to give 100%. The water phases given in Table 4 are combined with corresponding amounts of the polyol-in-wax preemulsion, thus W1 with 40% of a polyol-in-wax preemulsion, W2 with 50% of a polyol-in-wax preemulsion etc.

TABLE 5
Examples of active ingredient-containing suspensions

| | Ex. 3 | Ex. 4 | Ex. 5 | Ex. 6 | Ex. 7 | Ex. 8 | Ex. 9 | Ex. 10 |
|---|---|---|---|---|---|---|---|---|
| Water | W1 | W2 | W3 | W4 | W1 | W2 | W3 | W4 |
| phase | | | | | | | | |
| Wax | L1 | L2 | L3 | L4 | L1 | L4 | L2 | L3 |
| phase | | | | | | | | |
| Polyol | P1 | P2 | P3 | P4 | P2 | P3 | P1 | P4 |
| phase | | | | | | | | |

The active ingredient suspensions obtained in this way can be incorporated into all known, hydrous cosmetic or dermatologically acceptable systems, such as gels, W/O or O/W lotions, creams, sticks, impregnation media for wax, sprays and foams. This is preferably carried out at room temperature and after the last homogenization step, should one be required for the preparation of the system in question. The suspensions obtained in this way can be stored until used, it being advantageous to add a thickener or gel former which prevents the particles according to the invention from creaming.

In such a preparation, the active ingredient-containing polyol-in-wax particles according to the invention are present in suspended form in the water phase. The water phase can for its part be dispersed in an oil phase, although it is also possible for lipid particles or droplets of another composition to be dispersed in the same water phase. The overall preparation can also comprise active ingredients other than those present in the particles according to the invention. These active ingredients may either be lipophilic active ingredients or hydrophilic active ingredients. The preparation can also comprise the same active ingredient as within the particles according to the invention outside of these particles, for example in the aqueous phase of a cream, even if the active ingredient is not as stable in this medium as in the inside of the particles according to the invention.

1. A particle, comprising:
a solid coating primarily comprising at least one wax; and
a liquid core primarily comprising at least one polyol;
wherein the water content of the core is at most 50% by weight, based on the total weight of the core.

2. A plurality of particles according to claim 1, said particles having an average diameter of 0.5 to 50 μm.

3. A plurality of particles according to claim 2, said particles having an average diameter of 10 to 30 μm.

4. The particle according to claim 1, wherein the at least one polyol includes at least one polyol selected from the group consisting of unbranched polyhydroxyalkanes.

5. The particle according to claim 1, wherein the at least one polyol includes at least one polyol selected from the group consisting of glycerol, butylene glycol, dipropylene glycol and pentanediol.

6. The particle according to claim 1, wherein the at least one wax includes at least one wax having a melting point of from 35° C. to 80° C.

7. The particle according to claim 1, wherein the at least one wax includes at least one wax having a melting point of from 40° C. to 60° C.

8. The particle according to claim 1, wherein the at least one wax includes at least one hydrophobic wax.

9. The particle according to claim 1, wherein the at least one wax includes at least one paraffinic wax.

10. The particle according to claim 1, further comprising an interface between the coating and the core, said interface comprising TiO₂.

11. The particle according to claim 1, further comprising an emulsifier.

12. The particle according to claim 1, wherein the core further comprises at least one moisture-sensitive cosmetic or dermatological active ingredient.

13. The particle according to claim 12, wherein the at least one active ingredient includes at least one polar, water-insoluble active ingredient having a high crystallization tendency.

14. The particle according to claim 12, wherein the at least one polar, water-insoluble active ingredient includes at least one active ingredient selected from the group consisting of isoflavonoids, creatine, carnosine, pentacyclic triterpenes, flavonoids, green tea active ingredients, phytosterols, octadecenedioic acid, steroids and dihydroxyacetone.

15. The particle according to claim 14, wherein the at least one polar, water-insoluble active ingredient includes at least one isoflavonoid selected from the group consisting of genistein and daidzein.

16. The particle according to claim 14, wherein the at least one polar, water-insoluble active ingredient includes at least one pentacyclic triterpene selected from the group consisting of sericosides and ursolic acid.

17. The particle according to claim 14, wherein the at least one polar, water-insoluble active ingredient includes at least one flavonoid selected from the group consisting of rutin and quercetin.

18. The particle according to claim 14, wherein the at least one polar, water-insoluble active ingredient includes epigallocatechin gallate.

19. The particle according to claim 14, wherein the at least one polar, water-insoluble active ingredient includes at least one phytosterol selected from the group consisting of β-sitosterol and campesterol.

20. The particle according to claim 14, wherein the at least one polar, water-insoluble active ingredient includes at least one steroid selected from the group consisting of cholesterol and dehydroepiandrosterone.

21. The particle according to claim 12, wherein the at least one active ingredient includes at least one active ingredient that decomposes under the influence of moisture.

22. The particle according to claim 21, wherein the at least one active ingredient that decomposes under the influence of moisture includes at least one active ingredient selected from the group consisting of acetylcarnitine, acetylsalicylic acid, peptides consisting of 3-8 amino acids, enzymes, ascorbic acid and derivatives thereof.

23. The particle according to claim 22, wherein the at least one active ingredient that decomposes under the influence of moisture includes at least one enzyme selected from the group consisting of serine proteases, lipases, telomerases and DNA repair enzymes.

24. The particle according to claim 1, further comprising at least one antioxidant.

25. The particle according to claim 24, wherein the at least one antioxidant includes at least one antioxidant selected from the group consisting of ubiquinone, ubiquinol, vitamin E, EDTA, butylhydroxytoluene, α-tocopherol, and ascorbyl palmitate.

26. The particle according to claim 1, wherein the coating further comprises at least one photoprotective filter.

27. The particle according to claim 26, wherein the at least one photoprotective filter includes at least one photoprotective filler selected from the group consisting of butylmethoxydibenzoylmethane and anisotriazine.

28. A cosmetic or dermatological preparation comprising a plurality of particles according to claim 1.

29. A cosmetic or dermatological preparation comprising a plurality of particles according to claim 13.

30. A cosmetic or dermatological preparation comprising a plurality of particles according to claim 21.

31. A plurality of particles, the particles comprising:
a solid coating comprising at least one wax; and
a liquid core comprising at least one polyol;
wherein the water content of the core is at most 50% by weight, based on the total weight of the core.

32. A process for the preparation of a particle that comprises a solid coating comprising at least one wax and a liquid core comprising at least one polyol, wherein the water content of the core is at most 50% by weight, based on the total weight of the core, comprising the steps of:
(a) dissolving the wax in a volatile organic solvent to produce an organic phase;
(b) mixing the organic phase with an active ingredient-containing polyol phase to produce a polyol-in-solvent emulsion;
(c) mixing the polyol-in-solvent emulsion with a water phase to produce a polyol-in-solvent-in-water emulsion;
(d) removing the organic solvent to produce a polyol-in-wax-in-water suspension; and
(e) separating the particles from the polyol-in-wax-in-water suspension.

33. A process for the preparation of a particle that comprises a solid coating comprising at least one wax and a liquid core comprising at least one polyol, wherein the water content of the core is at most 50% by weight, based on the total weight of the core, comprising the steps of:
(a) mixing an active ingredient-containing polyol phase and a wax phase at a temperature above the melting temperature of the wax phase to produce a polyol-in-wax preemulsion;
(b) mixing the preemulsion and a water phase at a temperature above the melting temperature of the wax phase to produce a polyol-in-wax-in-water emulsion; and
(c) cooling the polyol-in-wax-in-water emulsion to a temperature below the melting temperature of the wax phase to produce a polyol-in-wax-in-water suspension and to produce the particles.

34. The process according to claim 33, further comprising the step of (d) separating the particles from the polyol-in-wax-in-water suspension.
